(19) United States
(12) Patent Application Publication
(10) Pub. No.: US 2025/0333704 A1
(43) Pub. Date: Oct. 30, 2025

(54) METHODS AND COMPOSITIONS RELATED TO ENGINEERED THERMOGENIC MICROVASCULARFRAGMENTS (MVFS)

(71) Applicant: Board of Regents, The University of Texas System, Austin, TX (US)

(72) Inventors: Maria Gonzales Porras, San Antonio, TX (US); Eric Brey, San Antonio, TX (US); Christopher Rathbone, San Antonio, TX (US); Katerina Stojkova, North Haven, CT (US); Francisca Acosta, San Antonio, TX (US)

(21) Appl. No.: 18/867,000

(22) PCT Filed: May 21, 2023
(86) PCT No.: PCT/US2023/023034

§ 371 Date: Nov. 18, 2024

Related U.S. Application Data
(60) Provisional application No. 63/343,563, filed on May 19, 2022.

(51) Int. Cl. C12N 5/077; A61K 35/35; A61P 31/04; C12N 9/64

(52) U.S. Cl. CPC C12N 5/0653; A61K 35/35; A61P 31/04; C12N 9/6489; C12N 2501/33; C12N 2501/39; C12N 2501/999; C12N 2513/00; C12N 2533/56

(57) ABSTRACT

Methods and compositions related to thermogenic adipose for study and therapeutics are described. Certain embodiments are directed to vascularized thermogenic adipose tissue developed using microvascular fragments (MVFs). MVFs are isolated and thermogenic adipose formed.

### PRIORITY PARAGRAPH

This Application is an International PCT application claiming priority to U.S. Provisional Application No. 63/343,563 filed May 19, 2022 which is incorporated herein by reference in its entirety.

### STATEMENT REGARDING FEDERALLY FUNDED RESEARCH

This invention was made with government support under SC1DK122578 awarded by the National Institutes of Health. The government has certain rights in the invention.

### BACKGROUND

Obesity is a chronic progressive disease and one of the leading causes of increased mortality among Americans, affecting 42.4% of adults in the United States (Saklayen, Curr Hypertens Rep 20 (2), 2018 12). Obesity is an established cause of Type 2 Diabetes Mellitus (T2D) and associated with numerous comorbidities, including cardiovascular disease (Poirier et al., Circulation 113(6) (2006) 898-918), certain cancers (Wolin et al., Oncologist 15(6), 2010 556-65), and hypertension (Eckel et al., Journal of clinical endocrinology and metabolism 96(6), 2011 1654-63). In addition to its serious health consequences, obesity presents an enormous financial burden on the health care system with expenditures of >$149 billion annually (Kim and Basu, Value in Health 19(5), 2016, 602-13). The leading cause of obesity is the disruption of energy equilibrium caused by excess energy consumption relative to energy dissipation (Romieu et al., Cancer Causes Control 28(3), 2017, 247-58; Unser et al., Biomaterials 75, 2016, 123-34).

Adipose tissue is essential for maintaining energy balance and a critical regulator of systemic metabolic function (Choe et al., Frontiers in Endocrinology 7(30), 2016). Excess energy in obese individuals leads to the expansion of white adipose tissue (WAT), a storage depot that also plays a role in the complex signaling processes regulating metabolic health (Barquissau et al., Mol Metab 5(5), 2016, 352-65). The majority of adipose tissue is WAT. In addition, brown adipose tissue (BAT) is a distinct depot which exhibits increased ability for energy expenditure and heat generation (Rosell et al., Am J Physiol Endocrinol Metab 306(8), 2014, E945-64). Activation of BAT is under investigation as a therapeutic target for combating the adverse metabolic consequences associated with obesity and its comorbidities; however, the small volume of BAT in adults could limit the ability to have significant and sustained impact on systemic metabolism (Yang et al., Tissue Eng Part A 23(7-8), 2017, 253-62; Kurylowicz and Puzianowska-Kuznicka, Int J Mol Sci 21(17), 2020; Srivastava et al., Front Physiol 10, 2019, 38; Kaisanlahti and Glumoff, J Physiol Biochem 75(1), 2019, 1-10; Mulya et al., Endocrinol Metab Clin North Am 45(3), 2016, 605-21; Lizcano and Vargas, Int J Endocrino/2016, 9542061).

A subpopulation of cells present in subcutaneous WAT depots can be induced to function as energy-burning cells (Cohen et al., Diabetes 64(7), 2015, 2346-51; Wu et al., Genes Dev 27(3), 2013, 234-50). These “beige” or “brite” cells exhibit similar morphological characteristics to brown fat, including multiocular lipid droplets and increased mitochondria, and increased metabolic activity as characterized by the process of mitochondrial uncoupling (Wu et al., Genes Dev 27(3), 2013, 234-50). Beige adipocytes exhibit upregulation of uncoupling protein 1 (UCP1). Upon activation by fatty acids, UCP1 uncouples oxidative phosphorylation in mitochondria, disrupting ATP synthesis and dissipating energy as heat (thermogenesis), a cycle also referred to as mitochondrial proton leak (Yang et al., Tissue Engineering Part A 23(7-8), 2017, 253-62). Intense catabolic activity occurs in brown and beige fat as it collects glucose, lipids, and oxygen at a high rate from the blood, aiding in glucose clearance and reducing the demand for insulin secretion. In animal models, an increase beige adipocytes has beneficial effects on whole-whole body metabolism, body weight, and glucose and lipid homeostasis (Kaisanlahti and Glumoff, Journal of Physiology and Biochemistry 75(1), 2019, 1-10; Wang et al., Science Translational Medicine 12(558), 2020, eaaz8664). “Browning” of subcutaneous WAT may be a critical target in the treatment and prevention of obesity, T2D, and other metabolic disorders (Kaisanlahti and Glumoff, Journal of Physiology and Biochemistry 75(1), 2019, 1-10; Cypess et al., New England Journal of Medicine 360(15), 2009, 1509-17; Loyd and Obici, Current Opinion in Clinical Nutrition & Metabolic Care 17(4), 2014, 368-72; Wankhade et al., BioMed Research International 2016, 2365609).

The rising awareness of brown/beige adipose tissue and its relation to adipose-related disorders has prompted increased interest in the development of in vitro models of functional beige adipose tissues to study metabolic conditions, identify therapeutic targets, and evaluate treatment options (Tharp and Stahl, Front Endocrinol (Lausanne) 6, 2015, 164; Vaicik et al., J Mater Chem B 3(40), 2015, 7903-11). While engineering models of adipose tissue has largely focused on WAT (McCarthy et al., Tissue Eng Part B Rev 26(6), 2020, 586-95; Murphy et al., BMC Biomed Eng 1, 2019; Unser et al., Biotechnol Adv 33(6 Pt 1), 2015, 962-79), 3D models of beige adipose tissue have been developed (Yang et al., Tissue Eng Part A 23(7-8), 2017, 253-62;Vaicik et al., J Mater Chem B 3(40), 2015, 7903-11; Mccarthy et al., Tissue Eng Part B Rev 26(6), 2020, 586-95; Klingelhutz et al., Sci Rep 8(1), 2018, 523; Tharp and Stahl, Frontiers in Endocrinology 6(164), 2015; Tharp et al., Diabetes 64(11), 2015, 3713-24; Harms et al., Cell Rep 27(1), 2019, 213-225.e5). However, the functional and structural relevance of these models to beige adipose tissue is limited.

Beige and white adipocyte precursors reside in a distinct perivascular niche in adipose tissues. This close proximity to vasculature is critical for both white adipose tissue expansion and the capacity for developing functional beige adipose tissues. This relationship may be particularly important in disease states where the function of the vasculature or precursor cells may be altered. Models that recreate this relationship can be used to gain new insight into beige adipose tissue development and function. Small vessel units that contain endothelial cells, basement membrane structure, and perivascular cells have been isolated from adipose tissue for applications in tissue engineering and regenerative medicine for years. However, it is not clear if these microvascular fragments (MVFs) retain functional adipocyte precursors following the isolation procedure.

Additional methods and compositions are needed to treat obesity and related pathologies.

### SUMMARY

Described herein is a solution to the problems of obesity and related pathologies, thermogenic compositions are produced having vascularized beige and white adipose tissue. The thermogenic components can be developed using microvascular fragments (MVFs). MVFs are isolated and thermogenic adipose is formed.

Certain embodiments are directed to thermogenic compositions comprising thermogenic cells in a vascularized hydrogel, the thermogenic cells being produced by culturing microvascular fragments (MVFs) in a thermogenic differentiation media. In certain aspects the thermogenic differentiation media comprises one or more of insulin, forskolin, dexamethasone, rosiglitazone, and T3. In certain aspects the differentiation media comprises: insulin; insulin and forskolin; insulin and dexamethasone; insulin and rosiglitazone; insulin and T3; insulin, forskolin, and dexamethasone; insulin, forskolin, and rosiglitazone; insulin, forskolin, and T3; insulin, dexamethasone, and rosiglitazone; insulin, dexamethasone, and T3; insulin, rosiglitazone, and T3; forskolin and dexamethasone; forskolin and rosiglitazone; forskolin and T3; forskolin, dexamethasone, and rosiglitazone; forskolin, dexamethasone and T3; forskolin, rosiglitazone, and T3; dexamethasone and rosiglitazone; dexamethasone and T3; dexamethasone, rosiglitazone, and T3; or insulin, forskolin, dexamethasone, rosiglitazone, and T3. In certain aspects the thermogenic cells are characterized by (1) multilocular lipid droplets, (2) increased number of mitochondria, (3) increased oxygen consumption rate, (4) increase in mitochondrial respiration, (5) increased metabolic activity compared to undifferentiated microvascular fragments, or any combination thereof. The composition can include thermogenic cell(s) expressing uncoupling protein 1 (UCP1) mRNA at a level that is at least 125, 150, 175, 200%, or more as compared to UCP1 mRNA expression in undifferentiated MVFs. Undifferentiated MVFs are those MVFs that have not been exposed to a differentiation media under conditions that develop thermogenic cells. The composition can include thermogenic cell(s) expressing cell death-inducing DNA fragmentation factor alpha-like effector A (Cidea) mRNA at a level that is at least 125, 150, 175, 200%, or more as compared to Cidea mRNA expression in undifferentiated MVFs. In certain aspects mRNA level(s) are determined by reverse transcriptase quantitative polymerase chain reaction (RT-qPCR), or equivalent methodology known in the art. The composition can include thermogenic cell(s) having UCPI and Cidea protein levels that are 125, 150, 175, 200, 300, 400%, or more as compared to protein levels in undifferentiated MVFs. In certain aspects the protein level is determined by Western blot or equivalent methodology known in the art. The composition can demonstrate a maximum respiration of the thermogenic cells as measured by oxygen consumption rate (pmol/min) after exposure to the ionophore carbonyl cyanide-ptrifluoromethoxyphenylhydrazone (FCCP) of at least 125, 150, 175, 200%, or more as compared to undifferentiated MVFs. The composition can demonstrate an isoproterenol stimulated lipolysis as measured by glycerol release of the thermogenic cells of at least 10.0, 12.5, or 25 mmol glycerol/cm². In still further aspects the composition can demonstrate a spare capacity of the thermogenic cells of at least 140, 150, 160, 180, 200%, or more as compared to undifferentiated MVFs. The measure of “spare capacity” is obtained by subtracting basal respiration from maximal oxygen consumption obtained by the titration of exposure to uncoupling agents such as carbonyl cyanide-p-trifluoromethoxyphenylhydrazone (FCCP). In certain aspects the hydrogel comprises 5 to 15 U/mL thrombin to 10 to 30 U/mL fibrinogen, preferably 10 U/mL thrombin and 20 U/mL fibrinogen. The differentiation of thermogenic cells comprises growing MVFs in a growth media for 0-14 days followed by growing the MVFs in a thermogenic differentiation media.

Other embodiments are directed to methods of producing a thermogenic composition comprising the steps of: mixing microvascular fragments (MVFs) isolated from a tissue in a hydrogel precursor solution; forming a MVF hydrogel by mixing the hydrogel precursor solution with fibrinogen and thrombin to form a MVF hydrogel; culturing the MVF hydrogel in a differentiation media. In certain aspects the differentiation media is a growth media containing one or more of insulin, forskolin, dexamethasone, rosiglitazone, or T3 forming a cultured thermogenic hydrogel. In certain aspects the differentiation media comprises: insulin; insulin and forskolin; insulin and dexamethasone; insulin and rosiglitazone; insulin and T3; insulin, forskolin, and dexamethasone; insulin, forskolin, and rosiglitazone; insulin, forskolin, and T3; insulin, dexamethasone, and rosiglitazone; insulin, dexamethasone, and T3; insulin, rosiglitazone, and T3; forskolin and dexamethasone; forskolin and rosiglitazone; forskolin and T3; forskolin, dexamethasone, and rosiglitazone; forskolin, dexamethasone and T3; forskolin, rosiglitazone, and T3; dexamethasone and rosiglitazone; dexamethasone and T3; dexamethasone, rosiglitazone, and T3; or insulin, forskolin, dexamethasone, rosiglitazone, and T3. The tissue can be an adipose tissue or other tissue. The method can further include isolating MVF by incubating a tissue (e.g., an adipose tissue) sample with collagenase forming a tissue digest; centrifuging the tissue digest forming a pellet and a floating layer; resuspending the pellet forming a suspension and filtering the suspension to collect MVFs in the filtrate forming collected MVFs. In certain aspects the thermogenic hydrogel is cultured for 5 to 15 days. The growth media, for example, can include Dulbecco's Modified Eagle Medium (DMEM), or an equivalent media known in the art, containing 20% Fetal Bovine Serum (FBS), 1% Penicillan-Streptomycin, and 0.2% MycoZap. The differentiation media contains one or more of 5 to 15 μg/ml Insulin, 5 to 15 μM Forskolin, 0.5 to 2 μM Dexamethasone, 0.5 to 2 μM Rosiglitazone, or 10 to 30 nM T3, preferably 10 μg/ml Insulin, 10 μM Forskolin, 1 μM Dexamethasone, 1 μM Rosiglitazone, or 20 nM T3. In certain aspects the differentiation media contains 10 μg/ml Insulin, 10 μM Forskolin, 1 μM Dexamethasone, 1 μM Rosiglitazone, and 20 nM T3. The cultured MVF hydrogel can be cultured for 2 to 21 days. The thermogenic MVF can be cultured in a maintenance media once differentiatited. The maintenance media can include, for example, DMEM/F12 containing 20% Fetal Bovine Serum (FBS), 1% Penicillan-Streptomycin, 0.2% MycoZap, 10 μg/ml Insulin, 10 μM Forskolin, 1 μM Rosiglitazone, and 20 nM T3.

Other embodiments are directed to an isolated thermogenic microvascular fragment (MVF) produced by the methods described herein. The thermogenic MVF can have an increased expression of thermogenic gene UCP-1 and/or increase in lipolysis upon exposure to isoproterenol, as compared to undifferentiated MVFs.

Certain embodiments are directed to methods of treating obesity or a metabolic disease comprising implanting the engineered thermogenic MVF into a subject in need thereof.

The term “adipocyte” refers to a cell that is specialized to synthesize and store fat. This term includes adipocytes with the properties representative of those present within white fat, and brown fat.

The term “adipose tissue” refers to a tissue that contains adipocytes that may or may not be accompanied by stromal cells, blood vessels, lymph nodes, tissue macrophages, and other cells and structures. The term includes tissue that is commonly referred to in the art as white adipose tissue (or white fat), or to brown adipose tissue (or brown fat). Adipose tissue is normally found in multiple sites within the body including, but not limited to subcutaneous adipose, visceral adipose, omental adipose, perirenal adipose, scapular adipose, inguinal adipose, adipose surrounding lymph nodes, medullary adipose, bone marrow adipose, pericardial adipose, retro-orbital adipose, and infrapatellar adipose. In the context of the present invention the term “adipose tissue” also refers to tissue that contains adipocytes or preadipocytes. The term further includes tissue that does not yet contain adipocytes but which is a precursor or anlage of such tissue.

The term “thermogenic adipocytes” refers to cells which have the characteristics of brown or beige fat, preferably human brown or beige fat. In particular, this term refers to adipocytes that express a “thermogenic” protein or “uncoupled protein-1” (UCP1, Gene ID: 7350), an uncoupling protein found in the mitochondria of brown adipocytes that generate heat by non-shivering thermogenesis, PPARγ2 (Peroxisome Proliferator-Activated Receptor gamma 2, Gene ID: 5468), CIDEA (Cell death-inducing DFFA-like effector A, Gene ID: 1149), LPL (Lipoprotein Lipase, Gene ID: 4023), ADIPOQ (Adiponectin C1Q and collagen domain containing, Gene ID: 9370), PGC1α (or PPARGC1A, Peroxisome Proliferator-Activated Receptor Gamma Coactivator 1 alpha, Gene ID: 10891), CEBPA (CCAAT/enhancer binding protein (C/EBP), alpha; Gene ID: 1050) and AP2 (or FABP4, Fatty acid binding protein 4; Gene ID: 2167).

The term “white adipose tissue cells” refers to cells present in white fat, preferably in human white fat, more preferably in adult human white fat. Two kinds of adipose tissue are found in mammals: white adipose tissue (WAT) and brown adipose tissue (BAT). WAT adipocytes contain few mitochondria and a single large fat droplet, which forces the nucleus to be squeezed into a thin rim at the periphery. They further secrete several hormones, including leptin and adiponectin. WAT adipocytes typically express RETN (Resistin, Gene ID: 56729). In mammals, WAT cells are located essentially beneath the skin (subcutaneous WAT), around internal organs (visceral WAT), in bone marrow (yellow bone marrow WAT) and in breast tissues.

As used herein, the term “subject” refers to an animal, preferably a mammal, and even more preferably a human, including an adult or a child.

“Metabolic Condition” or “Metabolic Disorder” or “Metabolic Syndrome” means a disease characterized by spontaneous hypertension, dyslipidemia, insulin resistance, hyperinsulinemia, increased abdominal fat, and/or an increased risk of coronary heart disease. As used herein, “metabolic condition” or “metabolic disorder” or “metabolic syndrome” shall mean a disorder that presents risk factors for the development of type 2 diabetes mellitus and cardiovascular disease and is characterized by insulin resistance and hyperinsulinemia and may be accompanied by one or more of the following: (a) glucose intolerance, (b) type 2 diabetes, (c) dyslipidemia, (d) hypertension and (e) obesity.

The term “obesity” refers to a medical condition in which excess body fat has accumulated to the extent that it may have a negative effect on health, leading to reduced life expectancy and/or increased health problems. A subject is considered obese when its body mass index (BMI), a measurement obtained by dividing a person's weight by the square of the person's height, exceeds 30 kg/m².

As used herein, the term “obesity-associated disease” or “obesity related pathologies” refers to diseases or disorders that have an increased likelihood to appear in obese subjects or are directly caused by obesity. In particular, this term may refer to type 2 diabetes, impaired glucose tolerance, insulin resistance, dyslipidemia, hypertension, and/or cardiovascular diseases.

As used herein, the term “treatment”, “treat”, or “treating” refers to any act intended to ameliorate the health status of patients such as therapy, prophylaxis, and/or retardation of the disease. In certain embodiments, such term refers to the amelioration of a disease or symptoms associated with a disease. In other embodiments, this term refers to minimizing the spread or worsening of the disease resulting from the administration of one or more therapeutic agents to a subject with such a disease.

In particular, the term “treatment of obesity or obesity-related disease” may refer to an increase of fat consumption, a loss of weight, a decrease of the insulin resistance, and/or improved glycemia.

By a “therapeutically efficient amount” is intended an amount of brown/beige adipocytes or engineered tissue administered to a subject that is sufficient to constitute a treatment as defined above.

The term “administering” means “delivering in a manner which is affected or performed using any of the various methods and delivery systems known to those skilled in the art. Administering can be performed, for example, intravenously, via implant, transdermally, intradermally, intramuscularly, subcutaneously, or intraperitoneally. Administering can also be performed, for example, once, a plurality of times, and/or over one or more extended periods.

An individual “at risk” may or may not have detectable disease and may or may not have displayed detectable disease prior to the treatment methods described herein. “At risk” denotes that an individual who is determined to be more likely to develop a symptom based on conventional risk assessment methods or has one or more risk factors that correlate with development of diabetes, metabolic syndrome, or obesity or an obesity-related disease, or a disease for which beige adipose administration provides a therapeutic benefit. An individual having one or more of these risk factors has a higher probability of developing diabetes, metabolic syndrome, obesity, or an obesity-related disease, than an individual without these risk factors. Examples (i.e., categories) of risk groups are well known in the art.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Immunofluoresence analysis of direct lean and diabetic microvascular fragment beige adipogenic differentiation after 14 days. (A) Schematic describing a first experiment and showing the different groups tested. (B) Representative confocal images of microvascular fragments grown in fibrin scaffolds and stained with GS-Lectin I to visualize vascular network formation and BODIPY to identify the presence of lipid droplets (full view scale bar=300 μm, inset scale bar=100 μm). (C) and (D) Quantitative analysis of vessel and lipid formation as determined with GS Lectin I (Lectin) or boron-dipyrromethene (BODIPY) accumulation, respectively. Quantification performed as a measurement of % well coverage within wells. (E) and (F) Quantification of lipid droplet size and number of lipids, respectively. Subcutaneous (SUBQ) fat was used as the source of microvascular fragments. Results are reported as mean±standard error of two experimental replicates (n=6 per experiment). *=p<0.05, **=p<0.01, ***=p<0.001, ****=p<0.0001. Lines across the experimental group indicate statistical significance relative to all groups.

FIG. 2. RT-qPCR analysis of main adipogenic, thermogenic, and angiogenic genes of microvascular fragments extracted from SUBQ fat of lean and diabetic rats after 14 days in culture. (A)-(C) Fold expression of several adipogenic genes, Fatty acid synthase (FAS), Adiponectin, and peroxisome proliferator-activated receptor gamma (PPRG). (D)-(E) Fold expression of several thermogenic genes, uncoupling protein 1 (UCP1) and cell death-inducing DNA fragmentation factor alpha-like effector A (Cidea). (F)-(H) Fold expression of several angiogenic genes, Fetal liver kinase 1 (FLK1), Angiopoietin-1 (ANGPT1), and vascular endothelial growth factor (VEGF). Results are reported as mean±standard error of two experimental replicates (n=4 per experiment). *=p<0.05, **=p<0.01, ***=p<0.001, ****=p<0.0001.

FIG. 3. Functional analysis of beige adipose tissue formation in direct adipogenic culture of lean and diabetic microvascular fragments. Microvascular fragments (MVF) from subcutaneous fat of lean and diabetic rats were directly exposed to white (WAM) or beige (BAM) adipogenic media for 14 days. At the end of the 14 days (A) Insulin stimulated glucose uptake (ISGU)±insulin, to stimulate glucose uptake and (B) lipolysis±isoproterenol, to stimulate lipolysis, was measured. (C)-(G) Oxygen Consumption Rate (OCR) trace was determined using a Seahorse XF96 Analyzer among the different groups, basal respiration, proton leak, maximal respiration, and spare capacity were calculated. Results are reported as mean +standard error of two experimental replicates (n=4 per experiment). *=p<0.05, **=p<0.01, ***=p<0.001, ****=p<0.0001.

FIG. 4. Immunofluoresence analysis of indirect lean and diabetic microvascular fragment beige adipogenic differentiation after 21 days. (A) Schematic describing the second experiment and showing the different groups tested. (B) Representative confocal images of microvascular fragments grown in fibrin scaffolds and stained with GS-Lectin I to visualize vascular network formation and BODIPY to identify the presence of lipid droplets (full view scale bar=300 μm, inset scale bar=100 μm). (C) and (D) Quantitative analysis of vessel and lipid formation as determined with GS Lectin I (Lectin) or boron-dipyrromethene (BODIPY) accumulation, respectively. Quantification performed as a measurement of % well coverage within wells. (E) and (F) Quantification of lipid droplet size and number of lipids, respectively. Subcutaneous (SUBQ) fat was used as the source of microvascular fragments. Results are reported as mean±standard error of two experimental replicates (n=6 per experiment). *=p<0.05, **=p<0.01, ***=p<0.001, ****=p<0.0001. Lines across the experimental group indicate statistical significance relative to all groups.

FIG. 5. RT-qPCR analysis of main adipogenic, thermogenic, and angiogenic genes of microvascular fragments extracted from SUBQ fat of lean and diabetic rats after 21 days in culture. (A)-(C) Fold expression of several adipogenic genes, Fatty acid synthase (FAS), Adiponectin, and peroxisome proliferator-activated receptor gamma (PPRG). (D)-(E) Fold expression of several thermogenic genes, uncoupling protein 1 (UCP1) and cell death-inducing DNA fragmentation factor alpha-like effector A (Cidea). (F)-(H) Fold expression of several angiogenic genes, Fetal liver kinase 1 (FLK1), Angiopoietin-1 (ANGPT1), and vascular endothelial growth factor (VEGF). Results are reported as mean±standard error of two experimental replicates (n=4 per experiment). *=p<0.05, **=p<0.01, ***=p<0.001, **=p<0.0001.

FIG. 6. Functional analysis of beige adipose tissue formation in indirect adipogenic culture of lean and diabetic microvascular fragments. Microvascular fragments (MVF) from subcutaneous fat of lean and diabetic rats were indirectly exposed to white (WAM) or beige (BAM) adipogenic media for 21 days. At the end of the 21 days (A) Insulin stimulated glucose uptake (ISGU)±insulin, to stimulate glucose uptake and (B) lipolysis±isoproterenol, to stimulate lipolysis, was measured. (C) Luminescence levels (proportional to H₂O₂levels) measured by the ROS-GLO assay in the MVF scaffolds exposed to growth media (GM-GM), white (GM-WAM) and beige (GM-BAM) differentiation media. (D)-(H) Oxygen Consumption Rate (OCR) trace was determined using a Seahorse XF96 Analyzer among the different groups, basal respiration, proton leak, maximal respiration, and spare capacity were calculated. Results are reported as mean±standard error of two experimental replicates (n=4 per experiment). *=p<0.05, **=p<0.01, ***=p<0.001, ****=p<0.0001.

FIG. 7. Immunofluorescence analysis of human microvascular fragment (hMVF) beige adipogenic differentiation after 14 days. (A) Representative phase microscopy picture of a hMVF after differentiation. (B) Representative confocal images of microvascular fragments grown in fibrin scaffolds and stained with GS-Lectin I to visualize vascular network formation and BODIPY to identify the presence of lipid droplets (scale bar=200 μm). (C) and (D) Quantitative analysis of vessel and lipid formation as determined with Lectin or BODIPY accumulation, respectively. (E) Quantification of lipid droplet size Results are reported as mean±standard error of two experimental replicates (n=6 per experiment). *=p<0.05, **=p<0.01,***=p<0.001, ****=p<0.0001.

FIG. 8. RT-qPCR analysis of main adipogenic and thermogenic genes of human microvascular fragments after 14 days in culture. (A) and (B) Fold expression of the adipogenic genes Adiponectin, and peroxisome proliferator-activated receptor gamma (PPRG). (C)-(F) Fold expression of several thermogenic genes, uncoupling protein 1 (UCP1), cell death-inducing DNA fragmentation factor alpha-like effector A (Cidea), peroxisome proliferator-activated receptor gamma coactivator 1-alpha (PGC-1α) and cytochrome C oxidase subunit VIIa polypedtide 1 (COX71A). Results are reported as mean±standard error of two experimental replicates (n=4 per experiment). *=p<0.05, **=p<0.01, ***=p<0.001. ****=p<0.0001.

FIG. 9. Functional analysis of beige adipose tissue formation in direct adipogenic culture of human microvascular fragments (hMVFs). hMVFs were directly exposed to white (WAM) or beige (BAM) adipogenic media for 14 days. At the end of the 14 days (A) lipolysis±isoproterenol, to stimulate lipolysis and (B) Insulin stimulated glucose uptake (ISGU)±insulin, to stimulate glucose uptake, was measured. (C) Oxygen Consumption Rate (OCR) trace was determined using a Seahorse XF96 Analyzer among the different groups, basal respiration, proton leak, maximal respiration, and spare capacity were calculated. Results are reported as mean±standard error of two experimental replicates (n=4 per experiment). *=p<0.05, **=p<0.01, ***=p<0.001, ****=p<0.0001.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

A number of medical and surgical approaches are under investigation that attempt exploit the enhanced metabolic activity of brown or beige adipose tissues as a treatment for obesity or metabolic disease (Samuelson and Vidal-Puig, Frontiers in Endocrinology 11(629), 2020). However, the limited volume of tissue available, poor understanding of adipose tissue physiology, and potential safety concerns of pharmacological agents limits successful translation of these approaches (Yang et al., Tissue Eng Part A 23(7-8), 2017, 253-62). Another option being considered is engineering of adipose tissues that are subsequently transplanted in an attempt to transform systemic metabolism. Transplanted BAT can decrease body fat, reverse insulin resistance, and improve overall metabolic function in animal models (Yang et al., Tissue Eng Part A 23(7-8), 2017, 253-62; Tharp and Stahl, Frontiers in Endocrinology 6(164), 2015; Stanford et al., J Clin Invest 123(1), 2013, 215-23; Liu et al., Endocrinology 156(7), 2015, 2461-9). Engineering adipose tissue constructs with metabolic function and structure similar to BAT (i.e., producing thermogenic compositions) using an easily accessible and potential autologous cell source can be a viable therapeutic intervention and could be used as a tool for studying BAT and its metabolic properties.

Microvascular fragments (MVF), isolated from adipose tissue, may provide for generating vascularized beige adipose tissue. MVF can be isolated from autologous visceral and subcutaneous adipose tissue depots harvested from adults using standard minimally-invasive procedures. Previous work has demonstrated that in addition to endothelial cell-lined capillaries, MVF are a source of adipose derived stem and progenitor cells, mural cells, and immune cells (Acosta et al., Tissue Eng Part A 26(15-16), 2020, 905-14; McDaniel et al., Journal of Surgical Research 192(1), 2014, 214-22; Laschke et al., Trends in Biotechnology 39(1), 2021, 24-33). While it is established that MVF can be used to generate white adipose tissue, it was unknown if MVF could be used to generate beige adipose tissue. Histologically, exposure of MVF to both white and beige adipogenic media resulted in lipid loading and adipogenic differentiation. Upon exposure to BAM conditions, the MVFs exhibited an increase in expression of the thermogenic genes UCP1 and Cidea. These results suggest that the isolated MVF contain precursor cells that can be induced to express markers of beige adipose tissue.

Adipose tissue serves as a major regulator of systemic metabolism, glucose homeostasis, insulin sensitivity, and energy regulator, in part, through coordination of lipogenesis and lipolysis. The improved function of beige adipose tissue over white adipose tissue is critical to its potential for therapeutic impact (Wang et al., Journal of lipid research 55(4), 2014, 605-24). Extensive functional analysis, including glucose uptake, lipolysis, and OCR were performed on the engineered adipose tissue. In general, the beige differentiated tissues exhibited enhanced function relative to white adipose tissues and controls. Insulin stimulated glucose uptake was generally higher in the BAM treated groups, which is consistent with brown adipose tissue (Mossenbock et al., PLOS ONE 9(10), 2014, e110428). The beige adipose tissues also exhibited increased lipolysis (Coolbaugh et al., Scientific Reports 9(1), 2019, 13600). Basal levels of lipolysis were increased in all adipogenic media groups; however, only BAM treated groups exhibited an increase in lipolysis with exposure to isoproterenol. For a more detailed analysis of the metabolic function of the tissue, mitochondrial bioenergetics were examined with OCR. Thermogenic adipose tissues are mitochondria rich, a primary reason for their distinguishing “brownish” appearance pathologically. The maximum rate of respiration was significantly higher in BAM treated groups. Proton leak, a measure of basal respiration not coupled to ATP production, also exhibited highest levels in BAM. Lastly, spare capacity was significantly highest among BAM treated groups, demonstrating that the greatest cell fitness or flexibility in responding to energetic demand was exhibited by beige adipose microtissues. In summary, engineered thermogenic adipose deriving from lean and diabetic MVFs, was able to portray several key functional measurements, including glucose uptake, lipolysis, and mitochondrial bioenergetics.

Activation of beige adipose tissue is expected to improve systemic metabolism in individuals with obesity and diabetes. However, there is limited understanding of the effects of diabetes on the potential formation and function of beige adipose tissues. MVF isolated from diabetic animals contained cells that could be induced to differentiate and express thermogenic markers at levels similar to MVF from lean animal models. Functionally, glucose uptake and lipolysis were similar in beige microtissues formed from both lean and diabetic rodent models. In the case of insulin resistance, obesity, and T2D, WAT mitochondria become dysfunctional contributing to oxidative stress and systemic inflammation leading to insulin resistance and the pathogenesis of T2D (Patti and Corvera, Endocr Rev 31(3), 2010, 364-95; Prasun, J Diabetes Metab Disord 19(2), 2020, 2017-22). Recently, brown adipose tissue from obese mice exhibit increased mitochondrial activity but accompanied with higher inflammation and oxidative damage relative to lean animals (Alcalá et al., Scientific Reports 7(1), 2017, 16082). Interestingly, beige microtissues generated from diabetic MVFs demonstrated the highest mitochondrial activity, significantly higher than MVF from lean animals exposed to BAM. For all other functional outcomes MVF from diabetic animals exhibited similar outcomes to MVF from lean animals. These results suggest that MVF from diabetic animals retain beige progenitor cells and these cells exhibit similar functional outcomes to lean when exposed directly to factors that induce beige adipogenesis.

Vascularization is essential for adipose tissue expansion and function (Huttala et al., Basic Clin Pharmacol Toxicol 123 Suppl 5, 2018, 62-71; Christiaens, Mol Cell Endocrinol 318(1-2), 2010, 2-9). Engineering functional beige adipose tissue that survives post implantation requires an extensive vascular network. Previously, it was shown that coordinating vessel network assembly and adipogenesis requires careful coordination of the timing (Acosta et al., Tissue Eng Part A 26(15-16), 2020, 905-14) and composition of differentiation media. Based on this knowledge, adipose tissue formation was examined following an initial 7-day phase of inducing network formation. Microtissues cultured in angiogenic conditions (GM and GM-GM) resulted in significantly lower vascular network formation with MVF from diabetic animals. In regard to the expression of genes associated with angiogenesis analysis, expression of VEGF, FLK1, and ANGPTI was lower in diabetic MVFs at 21 days. In the case of obesity, network formation may lag adipose tissue enlargement, leading to lower vascular densities and hypoxia (Corvera and Gealekman, Biochimica et Biophysica Acta (BBA)—Molecular Basis of Disease 1842(3), 2014, 463-72; Herold and Kalucka, Frontiers in Physiology 11(1861), 2021). This hypoxic environment is thought to lead to a disbalance and overexpression of pro-angiogenic and pro-inflammatory stimuli that can contribute to insulin resistance and diabetes development (Ye et al., Am J Physiol Endocrinol Metab 293(4), 2007, E1118-28). Overall, it is demonstrated through histological and angiogenic gene expression analysis that diabetic MVFs have a reduced vascularization capacity.

The utilization of MVFs as a source of adiposity and vascularization has previously been explored (Acosta et al., Tissue Eng Part A 26(15-16), 2020, 905-14; Acosta et al., Tissue Eng Part A, 2021) and Strobel et al. (Hannah et al., Biofabrication, 2021). An approach of “pre-sprouting” the MVFs for seven days prior to adipogenic media induction was employed for a total of 21 days in culture and groups deemed GM-WAM and GM-BAM for vascularized WAT and BAT, respectively. Histological analysis showed successful formation of lipid loaded cells in the presence of a vessel network. Vessel formation analysis showed no significant differences between medias; however, consistent with angiogenic conditions, MVF from diabetic animals exhibited lower network formation and expression of genes associated with angiogenesis. A general trend within all medias was observed in gene expression and network formation that was consistent with MVF from diabetic animals exhibiting a reduced capacity for angiogenesis. Interestingly, the studies demonstrate that adipogenic induction with either WAM or BAM does not disrupt angiogenesis. Instead, MVF serve as an excellent source for network formation even when exposed to adipogenic conditions.

Following 7-days of exposure to angiogenic media, the 3D culture system retained cells with the capacity for both white and beige adipogenesis. While lipid formation was similar between the groups, expression of adipogenic and thermogenic markers was generally lower in MVF from diabetic models in comparison to lean models. Interestingly, vascularized fat exhibited an overall improvement in function with little differences between diabetic and lean models. Insulin sensitivity was enhanced, particularly in groups cultured in BAM, lipolysis was improved and there was a significant reduction in the production of reactive oxygen species (ROS). Increase ROS levels during hyperglycemia or diabetes can result in cellular death, tissue damage, vascular dysfunction, and ultimately play a pivotal role in diabetic complications (Volpe et al., Cell Death Dis 9(2), 2018, 119; Zhou et al., Frontiers in Cardiovascular Medicine 8(131), 2021). Although the role of BAT in ROS regulation is not fully established, there is some indication that it could diminish ROS production and oxidative damage (Shabalina et al., Biochimica et Biophysica Acta (BBA)—Bioenergetics 1837(12), 2014, 2017-30). Beige vascularized fat (GM-BAM) also exhibited the highest maximal respiration and spare capacity. Overall, utilizing an indirect approach allowed for the generation of vascularized fat from both lean and diabetic MVF sources. While vascularization and expression of thermogenic genes was lower with MVF isolated from diabetic animals, the metabolic or functional performance of the beige adipose tissue was maintained and dramatically enhanced over white adipose tissue.

The studies described below show that MVF from a rodent model of diabetes exhibit the capacity for generating vascularized beige adipose tissue with enhanced function. In the work described, subcutaneous adipose tissue was used for MVF isolation. The ease by which subcutaneous adipose tissue can be isolated using minimally invasive procedures allows for the potential to translate into application using autologous tissues. In addition, subcutaneous adipose formation exhibits a greater capacity for beige adipose formation. T2D and other metabolic complications are often associated with the accumulation of visceral fat.

### I. Microvascular Fragment Culturing

In practicing the methods disclosed herein, the cells that are used to generate thermogenic compositions may be obtained from adipose tissue. Adipose tissue can be obtained by any method known to a person of ordinary skill in the art. For example, adipose tissue may be removed from a patient by suction-assisted lipoplasty, ultrasound-assisted lipoplasty, or excisional lipectomy. In addition, the procedures may include a combination of such procedures, such as a combination of excisional lipectomy and suction-assisted lipoplasty. The tissue extraction should be performed in a sterile or aseptic manner to minimize contamination. Suction-assisted lipoplasty may be desirable to remove the adipose tissue from a human patient as it provides a minimally invasive method of collecting tissue with minimal potential for cell damage that may be associated with other techniques, such as ultrasound-assisted lipoplasty. Adipogenic cells can be obtained from adipose tissue as described in the art. Most methods apply enzymatic digestion of washed adipose tissue fragments followed by centrifugation to separate buoyant adipocytes and debris from the non-buoyant cell fraction.

For suction-assisted lipoplastic procedures, adipose tissue can be collected by insertion of a cannula into or near an adipose tissue depot present in the patient followed by aspiration of the adipose into a suction device. In one embodiment, a small cannula may be coupled to a syringe, and the adipose tissue may be aspirated using manual force. Using a syringe or other similar device may be desirable to harvest relatively moderate amounts of adipose tissue (e.g., from 0.1 ml to several hundred milliliters of adipose tissue). Procedures employing these relatively small devices have the advantage that the procedures can be performed with only local anesthesia, as opposed to general anesthesia. Larger volumes of adipose tissue above this range (e.g., greater than several hundred milliliters) may require general anesthesia at the discretion of the donor and the person performing the collection procedure. When larger volumes of adipose tissue are desired to be removed, relatively larger cannulas and automated suction devices may be employed in the procedure.

Excisional lipectomy procedures include, and are not limited to, procedures in which adipose tissue-containing tissue (e.g., skin) is removed as an incidental part of the procedure; that is, where the primary purpose of the surgery is the removal of tissue (e.g., skin in bariatric or cosmetic surgery) and in which adipose tissue can be removed along with the tissue of primary interest (e.g., extraction of perirenal or omental adipose during abdominal surgery). Subcutaneous adipose tissue may also be extracted by excisional lipectomy in which the adipose tissue is excised from the subcutaneous space without concomitant removal of skin. Harvesting adipose tissue via excisional lipectomy of the inguinal fat depot is contemplated when using adipose tissue from mice. The adipose tissue that is removed from a patient or animal can be collected into a device for further processing.

The amount of tissue collected will be dependent on a number of variables including, but not limited to, the body mass index of the donor, the availability of accessible adipose tissue harvest sites, concomitant and pre-existing medications and conditions (such as anticoagulant therapy), and, in the case of research animals, the number of donors selected.

The intact tissue fragments are then disaggregated using any conventional techniques or methods, including mechanical force (mincing or shear forces), enzymatic digestion with single or combinatorial proteolytic enzymes, such as collagenase, trypsin, lipase, liberase H1, or members of the Blendzyme family as disclosed in U.S. Pat. No. 5,952,215, and pepsin, or a combination of mechanical and enzymatic methods. For example, the cellular component of the intact tissue fragments may be disaggregated by methods using collagenase-mediated dissociation of adipose tissue, similar to the methods for collecting microvascular endothelial cells in adipose tissue, as disclosed in U.S. Pat. No. 5,372,945. Additional methods using collagenase that may be used in practicing the invention are disclosed in U.S. Pat. No. 5,952,215. Similarly, a neutral protease may be used instead of collagenase, as disclosed in Twentyman, et al. (Twentyman, et al., 1980, Cancer Lett. 9(3): 225-8). Furthermore, methods may employ a combination of enzymes, such as a combination of collagenase and trypsin or a combination of an enzyme, such as trypsin, and mechanical dissociation.

Separation of the cells in the suspension may be achieved by buoyant density sedimentation, centrifugation, elutriation, filtration, differential adherence to and elution from solid phase moieties, antibody-mediated selection, differences in electrical charge; immunomagnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295.

In one example, the tissue is washed with sterile buffered isotonic saline and incubated with collagenase at a collagenase concentration, a temperature, and for a period of time sufficient to provide adequate disaggregation.

In one embodiment, solutions contain collagenase at concentrations from about 10 μg/ml to about 50 μg/ml and are incubated at from about 30° C. to about 38° C. for from about 20 minutes to about 60 minutes. These parameters will vary according to the source of the collagenase enzyme, optimized by empirical studies, in order to confirm that the system is effective at extracting the desired cell populations in an appropriate time frame. A particular preferred concentration, time and temperature is 20 μg/ml collagenase (mixed with the neutral protease dispase; Blendzyme 1, Roche) and incubated for 45 minutes at about 37° C. An alternative preferred embodiment applies 0.5 units/mL collagenase (mixed with the neutral protease thermolysin; Blendzyme 3) and digests tissue for approximately 20 minutes.

Following disaggregation the active cell population can be washed/rinsed to remove additives and/or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously.

In addition to the foregoing, there are many post-wash methods that may be applied for further purifying the active cell population. These include both positive selection (selecting the target cells), negative selection (selective removal of unwanted cells), or combinations thereof. Post-processing manipulation may also include cell culture or further cell purification.

### II. Transplantation of Cells

Delivery of cells, e.g., human cells, into the subcutaneous space, into muscle, into spaces between muscle groups, within the abdominal and thoracic cavities, or within the bone are contemplated. Methods for delivery are known in the art and described in the literature. Certain cells with the capacity to differentiate into adipocytes are capable of migrating to locations such as the medullary cavity of bone, to the spleen, or to other tissues (including perivascular tissue) following intravenous administration. Therefore, delivery via intravascular routes of administration is also contemplated. Cells can be delivered in suspension, in semi-solid carriers such as hydrogels, or in (or on) scaffolds such as woven and non-woven fiber-based scaffolds, sponges, and other highly porous structures. In embodiments such scaffolds can be engineered to include chemical or surface modifications that enhance attachment, proliferation, and/or differentiation and maturation of the cells into adipose tissue-like tissue.

Sponge-like scaffolds can be generated from thermoplastic substrates such as polyglycolide (PGA). Thermal compression of salt particles of defined size (generated by passing particles through sieves to generate a fixed size range) into preformed polymeric sheets followed by elution of the salt particles in an aqueous solvent generates scaffolds with high porosity, high pore interconnectivity, controllable pore size, and structural integrity. Similar scaffolds can be generated by a solvent-casting/freeze-drying/particulate leaching method and by other methods that are known in the art. These scaffolds can then be washed, sterilized, and seeded with cells (fresh cells, cultured cells, or cultured/predifferentiated cells) that can be implanted by injection or surgical insertion or other means. This approach provides a solid substrate to which the cells can attach and proliferate and/or differentiate. It further creates a space that is largely protected from forces generated by movement of skin against underlying structures, muscle against muscle, or in the intraperitoneal cavity. Similarly, a scaffold-like structure which simply maintains a protected space in which the implant can form in a hydrogel, scaffold, or other medium is also within the scope of the present invention. The chemical and physical properties of the polymer should be compatible with the biology of the cells and of the host.

### A. Injection

Cells, such as human cells, in suspension or loaded onto small scaffolds (for example beads or microbeads) can be implanted into host animals by injection. Implantation can be into the subcutaneous space, the peritoneal cavity, the medullary cavity of bone, or into other space (such as intramuscular or under the kidney capsule). Cells may be delivered on a bead-like or particulate scaffold using injection provided that a sufficiently large gauge needle is used such that the beads do not block the needle or that the application of injection force does not apply a degree of shear force to the beads or cells resulting in significant reduction of the integrity of the scaffold or viability of the cells. Cells may be injected in a simple aqueous solution such as physiologic saline or in an injectible hydrogel such as collagen or Matrigel. Many other injectible carrier materials are known in the art and have been described in the literature, e.g., peptide-based scaffolds, self-assembling materials, and synthetic polymers.

### B. Surgical Implantation

Cells, e.g., human cells may also be delivered to a host animal by surgical implantation. For example, cells may be seeded onto woven, non-woven, or molded scaffolds of defined porosity that are then placed within the desired site. For example, sponge-like scaffolds can be generated from thermoplastic substrates such as polylactide-coglycolide (PLGA). Thus, thermal compression of salt particles of defined size (generated by passing particles through sieves to generate a fixed size range) into preformed polymeric sheets followed by elution of the salt particles in an aqueous solvent generates scaffolds with high porosity, high pore interconnectivity, controllable pore size, and structural integrity. Similar scaffolds can be generated by a solvent-casting/freeze-drying/particulate leaching method. Seeded scaffold can be implanted directly or subjected to further culture in regular medium or in medium that induces adipogenesis.

A combination of injection and surgical implantation may also be applied. For example, injection under the kidney capsule following surgical visualization of the injection site is contemplated.

### III. Conditions to be Treated

The disclosed compositions and methods are particularly useful for treating a subject having a disease, disorder, condition or symptom or comorbidity thereof associated with aging or increased age, metabolic disorders such as insulin resistance or diabetes, vascular disease, heart disease, atherosclerosis, dyslipidemia, liver steatosis, obesity or excessive weight gain, loss of physical activity, loss of endurance, loss of skeletal muscle strength, and loss of skeletal muscle mass. In some embodiments, the compositions and methods can reduce aging or pre-mature aging, increase longevity, increase lifespan, or combination thereof in a subject. The compositions can be used to increase mitochondrial biogenesis and oxidative metabolism.

### A. Metabolic Disorders

The disclosed compositions and methods are useful for treating one or more symptoms or comorbidities of a metabolic disorder, including, but not limited to, insulin resistance, Type 1 or 2 diabetes mellitus, insulin insensitivity, impaired fasting glycaemia, impaired glucose tolerance (IGT), dysglycemia, dyslipidemia, hypertriglyceridemia, hyperglyceridemia, dyslipoproteinemia, hyperlipidemia, hypercholesterolemia, hypolipoproteinemia, and metabolic syndrome.

### 1. Insulin Resistance and Diabetes

In some embodiments the disclosed compositions are used to treat or prevent insulin resistance or diabetes. Insulin resistance and diabetes can be diagnosed using an oral glucose tolerance test (OGTT). Typically, a fasting patient takes a 75 gram oral dose of glucose. Blood glucose levels are then measured over the following 2 hours. After 2 hours a glycemia less than 7.8 mmol/L (140 mg/dl) is considered normal, a glycemia of between 7.8 to 11.0 mmol/dl (140 to 197 mg/dl) is considered as Impaired Glucose Tolerance (IGT) and a glycemia of greater than or equal to 11.1 mmol/dl (200 mg/dl) is considered diabetes mellitus. An OGTT can be normal or mildly abnormal in simple insulin resistance. A fasting serum insulin level of greater than approximately 60 pmol/L is also considered evidence of insulin resistance.

In some embodiments, the disclosed compositions reduce or decrease fasting blood glucose level, insulin level, or combinations thereof, or to reduce, decrease, or delay a rise in fasting blood glucose level, insulin level, or combinations thereof over time. In some embodiments, the compositions disclosed herein delay a rise in fasting blood glucose level, insulin level, or combinations thereof that can occur over time in subjects with high fat diets, little or no exercise, hereditary mutations, hormone changes, advanced age (i.e., becoming elderly), increasing weight or other factors that put them at risk for insulin resistance or diabetes.

### 2. Metabolic Syndrome

In some embodiments the metabolic disorder is metabolic syndrome, which typically includes a finding of at least two, preferably three or more of the following symptoms: blood pressure equal to or higher than 130/85 mmHg; fasting blood sugar (glucose) equal to or higher than 100 mg/dL; large waist circumference (length around the waist): Men—40 inches or more, Women—35 inches or more; low HDL cholesterol: Men—under 40 mg/dL, Women—under 50 mg/dL, Triglycerides equal to or higher than 150 mg/dL.

In some embodiments, a method for treating or inhibiting the progression of a metabolic disorder or disease in a subject in need thereof by administering to the subject an effective amount of a disclosed composition. The subject can display one or more symptoms selected from the group consisting of excessive appetite relative to healthy subjects, elevated blood glucose levels relative to healthy subjects, increased glucose sensitivity relative to healthy subjects, increased glycosylated protein levels relative to healthy subjects, elevated insulin levels relative to healthy subjects, decreased insulin sensitivity relative to healthy subjects, increased blood triglyceride levels relative to healthy subjects, increased blood cholesterol levels relative to healthy subjects, increased blood free fatty acid levels relative to healthy subjects, or a combination thereof. The metabolic disorder or disease can be selected from the list consisting of prediabetes, impaired fasting glycaemia, impaired glucose tolerance (IGT), dysglycemia, insulin resistance, hypertriglyceridemia, hyperglyceridemia, stroke, arteriosclerotic vascular disease (ASVD), dyslipoproteinemia, hypolipoproteinemia, and hyperlipidemia or hypercholesterolemia.

Comorbidities of metabolic disorders include heart disease, vascular disease, atherosclerosis, diabetes, heart attack, kidney disease, nonalcoholic fatty liver disease, peripheral artery disease, and stroke.

In some embodiments, the disclosed compositions are used to prevent, improve, reduce, delay, or improve one or more symptoms or comorbidities of metabolic disorder.

Methods of treating a metabolic disorder can also include dietary modifications such as reduced fat, increased fruits, vegetables, and whole-grain products, increase fish or fish oils; increased exercise; weight loss; managing blood pressure and blood sugar; and not smoking. Combination therapies can include administration of the compositions disclosed herein in combination with a second therapeutic agent that is known in the art for treating insulin resistance, Type 1 or 2 diabetes mellitus, high cholesterol, high blood lipids, metabolic syndrome, or a symptom of comorbidity thereof. For example, the compositions can be administered in combination with insulin or a cholesterol-lowering drug. Cholesterol lowering drugs include, but are not limited to, statins such as atorvastatin (Lipitor), simvastatin (Zocor), lovastatin (Mevacor), pravastatin (Pravachol), and rosuvastatin (Crestor).

### B. Weight Gain and Obesity

In some embodiments, the disclosed compositions are used to reduce or decrease, total body weight in a subject. The disclosed compositions can also be used to reduce, decrease, or delay a rise in total body weight over time. In some embodiments, the compositions disclosed herein delay a rise in total body weight, for example, that which can occur over time in subjects with high fat diets, little or no exercise, hereditary mutations, hormone changes, advancing age (i.e., elderly), diabetes, high cholesterol or high triglycerides.

The disclosed compositions and methods can be used for treating or preventing obesity or one or more symptoms or comorbidities thereof.

In some embodiments the subject is a healthy individual of normal weight, or is already overweight, and any additional weight gain could result in obesity or obesity-associate comorbidities. Body Mass Index is a standardized method of determining a subject's weight category using a calculus that is known in the art. A subject can be, for example, underweight: BMI of less 18.5; normal weight: BMI of 18.5-24.9; overweight: BMI of 25-29.9; or obese: BMI of 30 or greater. Therefore, in preferred embodiments, the disclosed compositions are useful for treating or preventing weight gain in a subject with a normal BMI, an overweight BMI, or an obese BMI. For example, the disclosed compositions can be used to treat or prevent weight gain in a subject with a BMI of about 25, 26, 27, 28, 29, 30, or more.

In some embodiments, the subject consumes less food, for example, 0.5%, 1%, 2%, 3%, 4%, 5%, 10%, 15%, 20%, 25%, or 50% less food, over time while being administered the compositions.

### IV. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Results

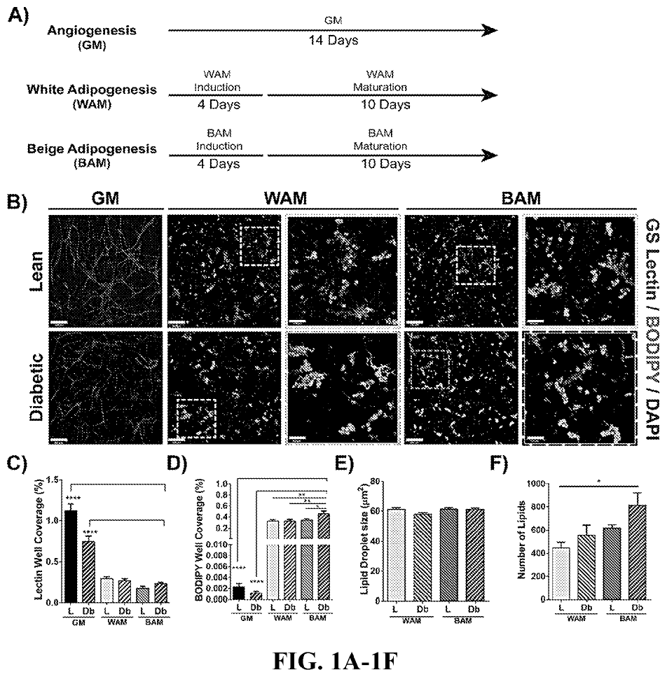

Expression of white and beige adipogenic markers by MVF isolated from lean and diabetic rats. MVF were isolated from the subcutaneous adipose fat of lean and diabetic rats. There were no qualitative differences in the amount of quality of the MVF from diabetic rats relative to lean. The MVF were suspended in fibrin gels and grown for 14 days in either GM, WAM, or BAM conditions (FIG. 1A). Both lean and diabetic MVF scaffolds grown in GM exhibited an interconnected network of lectin-positive cells (FIG. 1B). When MVFs were directly exposed to WAM or BAM differentiation media, they underwent adipogenic differentiation exhibited by BODIPY stained lipid droplets present with MVFs isolated from both lean and diabetic animals. Qualitatively, lipid loading appeared to be greater in MVF from diabetic relative to lean and with exposure to BAM relative to WAM (FIG. 1B).

Quantitative analyses were performed on three-dimensional confocal images of the fibrin scaffolds to compare vessel network and lipid droplet formation between experimental groups. Network formation, measured as a percent well coverage of GS lectin I stain, was highest with exposure to GM, having significantly greater lectin staining in comparison to WAM or BAM (FIG. 1C). The highest level of angiogenesis was seen in the lean GM group, which was statistically significant in comparison to all other groups, including the diabetic MVF scaffolds grown in GM (1.1±0.08 vs. 0.75±0.06%, p<0.0001). Similarly, the percent well coverage of lipid droplets (BODIPY stain) was quantified as a measure of adipogenesis (FIG. 1D). The scaffolds grown in GM exhibited negligible lipid formation in both lean and diabetic groups (0.002±0.0006% and 0.001±0.0003% respectively). On the other hand, there was a statistically significant increase in BODIPY staining in the groups treated with adipogenic media compared to GM controls. Lean and diabetic WAM groups were at similar levels in lipid droplet formation (0.33±0.03% and 0.32±0.03%, respectively) and not different from the lean BAM group (0.34±0.03%). Interestingly, MVF isolated from diabetic animals treated with BAM exhibited significantly greater lipid loading than any other condition (Db BAM vs. L. WAM, Db WAM and I BAM: 0.46±0.05% vs 0.33±0.03%, p=0.007, vs. 0.32±0.03%, p=0.006, vs. 0.34±0.03%, p=0.01, respectively; FIG. 1C). In addition, the size and number of the lipid droplets formed in each group was analyzed (FIG. 1E-F). There were no differences in the lipid droplet size between all experimental groups, but there was a significant difference in the number of lipid droplets between the lean WAM and diabetic BAM group (447±49 vs. 815±107, p=0.01, respectively), consistent with the results from percent area.

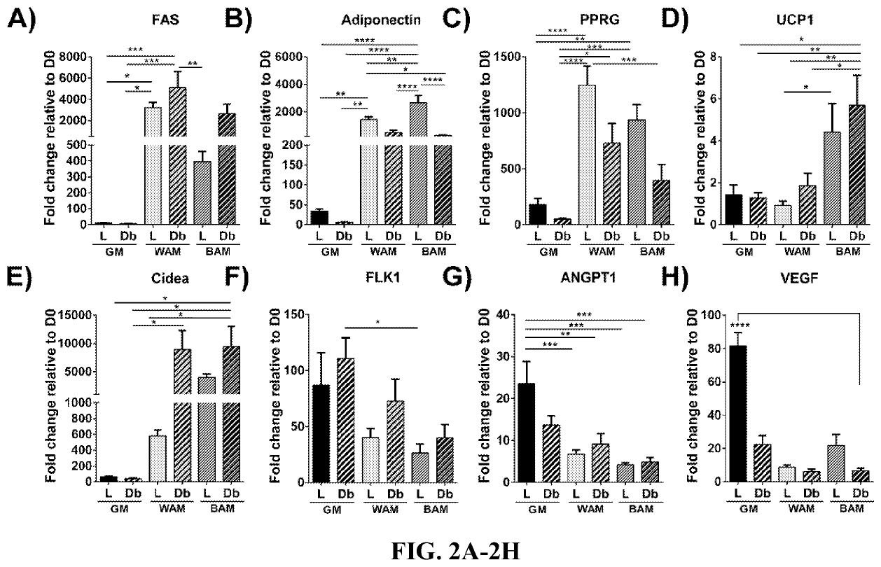

The expression of adipogenic and thermogenic markers was examined by RT-qPCR. Genes associated with adipogenesis, including adiponectin, fatty acid synthase (FAS), and peroxisome proliferator-activated receptor gamma (PPRG), were greater in WAM and BAM groups in comparison to controls (GM, FIG. 2A-2C). FAS, an enzyme that catalyzes the formation of long-chain fatty acids in adipose tissue, was expressed MVFs from both lean and diabetic groups cultured in WAM and BAM conditions with the highest expression levels in the diabetic MVF group grown in WAM (FIG. 2A). Adiponectin, a protein expressed primarily in mature adipocytes, was also expressed in both lean and diabetic MVF scaffolds exposed to WAM and BAM conditions. Adiponectin expression levels were higher in the lean groups in both WAM and BAM conditions, with the lean BAM group having significantly higher expression levels in comparison to all experimental groups (p<0.0001, FIG. 2B). PPRG, a protein that regulates adipocyte differentiation, exhibited similar trends with significantly greater expression levels in lean MVFs cultured in WAM and BAM compared to control groups (p<0.0001 and p<0.001, respectively). There were no significant differences in PPRG expression levels between lean and diabetic MVF exposed to either WAM or BAM conditions; however, expression levels demonstrated a trend towards lower levels in the MVFs from diabetic animals (FIG. 2C).

The thermogenic characteristics of the systems were first examined by evaluating expression of uncoupling protein 1 and cell death-inducing DNA fragmentation factor alpha-like effector A (UCP1 and Cidea, respectively), genes involved in thermogenesis that are upregulated in brown and beige adipose tissues. The expression of UCP1, a mitochondrial protein involved in thermogenic respiration, was increased in both lean and diabetic BAM MVF groups relative to GM and WAM groups. UCP1 was significantly higher in lean BAM relative to lean WAM (p=0.036). Interestingly, expression of UCP1 was highest in the diabetic BAM group (˜6 fold), with levels significantly higher than lean and diabetic WAM and GM groups (p<0.05). There was no significant difference in UCP1 levels between lean and diabetic MVFs exposed to BAM conditions (FIG. 2D). Cidea, a regulator of the thermogenic function in brown/beige fat, was increased in both WAM and BAM diabetic groups. MVFs from diabetic animals models exhibited significantly higher expression of Cidea in comparison to GM groups and the lean WAM group (p<0.05, FIG. 2E) (Jash et al., iScience, 20, 73-89, 2019). These results indicate that MVFs isolated from both lean and diabetic animals contain cells that can be induced to express markers of beige or brown adipose tissue.

In addition to genes associated with adipogenesis, genes involved in angiogenesis were evaluated. Levels of fetal liver kinase 1 (FLK1), an early angiogenic marker, was higher in all experimental groups relative to day 0. FLKI was slightly greater in diabetic groups cultured in GM, WAM, and BAM conditions relative to the lean groups, but the differences were not statistically significant (FIG. 2F). Angiopoietin-1 (ANGPT1), a late angiogenic marker, exhibited the highest expression levels in MVFs from lean animal models and exposed to GM, with statistical significance relative to all WAM and BAM cultured conditions (p<0.005, FIG. 2G). Expression of vascular endothelial growth factor (VEGF), a key promotor of angiogenesis, was increased in all experimental groups relative to day 0 with the lean GM group (˜81-fold increase), significantly higher than all other conditions (p<0.0001, FIG. 2H). In addition, VEGF expression levels in MVF from diabetic animals in all growth conditions were lower relative to the lean MVFs. These results suggest that MVFs from diabetic rats may exhibit a decreased capacity for angiogenesis.

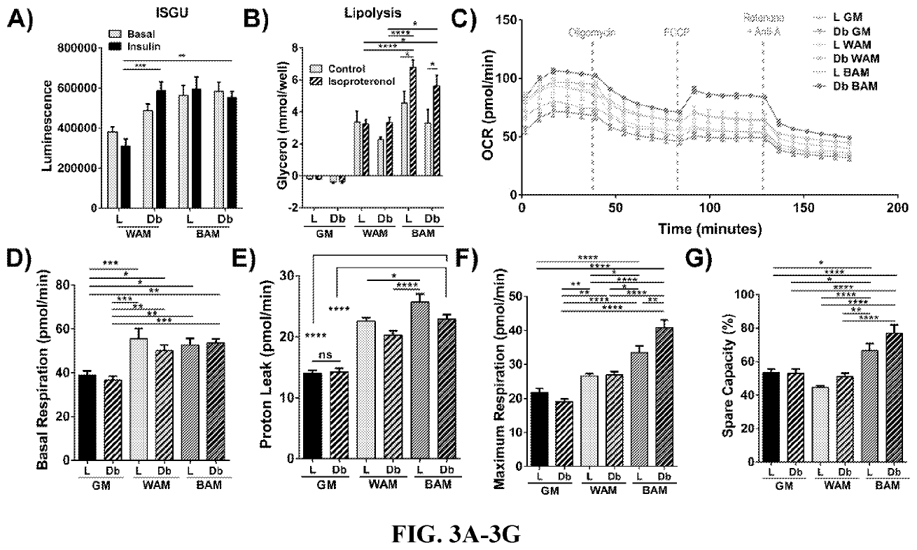

Functional assessment of engineered adipose tissues. Function of the engineered tissues was first evaluated based tissue glucose uptake under basal conditions and upon insulin stimulation (FIG. 3A). There was a significant different in insulin-stimulated glucose uptake (ISGU) in lean and diabetic BAM groups as well as diabetic WAM relative to lean MVFs in WAM conditions (p=0.0004, p=0.005 and p=0.0005, respectively).

Lipolysis was used as an additional measure of adipose tissue function. Mature adipocytes in culture respond to CAMP analogs, such as isoproterenol, that stimulate lipolysis which can be measured by the level of glycerol released in the culture media (FIG. 3B). There were no differences in glycerol levels between control and isoproterenol stimulated lean and diabetic MVF groups grown in GM. On the other hand, MVFs from diabetic animals in WAM conditions and lean and diabetic MVFs in BAM conditions exhibit an increased release of glycerol relative to the controls following stimulation with isoproterenol. Further, there was a significant increase in lipolytic activity in both lean and diabetic MVFs in BAM conditions relative to their respective controls (6.8±0.5 vs. 4.6±0.7 mmol/well. p=0.02 and 5.6±0.7 vs. 3.3±0.9 mmol/well, p=0.01). In addition, both the lean and diabetic MVFs in BAM had a significantly greater level of glycerol compared to all WAM conditions when induced with isoproterenol (p<0.0001 and p<0.05, respectively).

The unique metabolic profile of brown/beige adipose tissue is critical for its potential as a therapeutic approach to the treatment of diabetes and obesity. Therefore, OCR was used to further evaluate metabolic activity in all experimental groups. OCR was measured while applying oligomycin (inhibits ATP synthase), FCCP (stimulates maximal respiration), and rotenone/antimycin A (inhibits ETC and enables the calculation of nonmitochondrial respiration), in sequence over the course of 3 hours (FIG. 3C). Both lean and diabetic MVFs exposed to WAM and BAM conditions had a significant increase in basal respiration rate relative to lean and diabetic GM groups. However, there was no difference in the basal respiration rate between the lean and diabetic MVFs exposed to WAM or BAM conditions (FIG. 3D). Proton leak, a measure of uncoupled respiration, was highest in lean MVFs exposed to BAM (25.7±1.3 pmol/min, FIG. 3E) with a significant difference in compairson to lean and diabetic GM (13.9±0.5 pmol/min and 14.2±0.6 pmol/min, p<0.0001) and lean and diabetic WAM (22.5±0.5 pmol/min, p=0.03 and 20.3±0.7 pmol/min, p<0.0001). No differences were observed between lean MVF and diabetic MVF in BAM conditions. Maximum respiration was highest in lean and diabetic MVFs in BAM (33.5±1.9 and 40.8±2.3 pmol/min) which was significantly different from WAM (26.7±0.7 and 26.9±0.9 pmol/min p<0.05 and p<0.0001, respectively) and GM (lean, 21.7±1.3 and diabetic, 19.1±0.7 pmol/min, p<0.0001, FIG. 3F). Interestingly, maximum respiration of diabetic MVFs was significantly higher than lean MVFs grown to BAM conditions (p=0.006). The spare capacity, a measure of the ability of cells to achieve maximum respiration, was also higher in both lean and diabetic MVFs n BAM with a significantly higher percent in diabetic MVFs in BAM relative to (77±5%, p<0.0001 FIG. 3G). These findings correlate with the increased mRNA expression of UCP1 in the diabetic MVF scaffolds in BAM conditions.

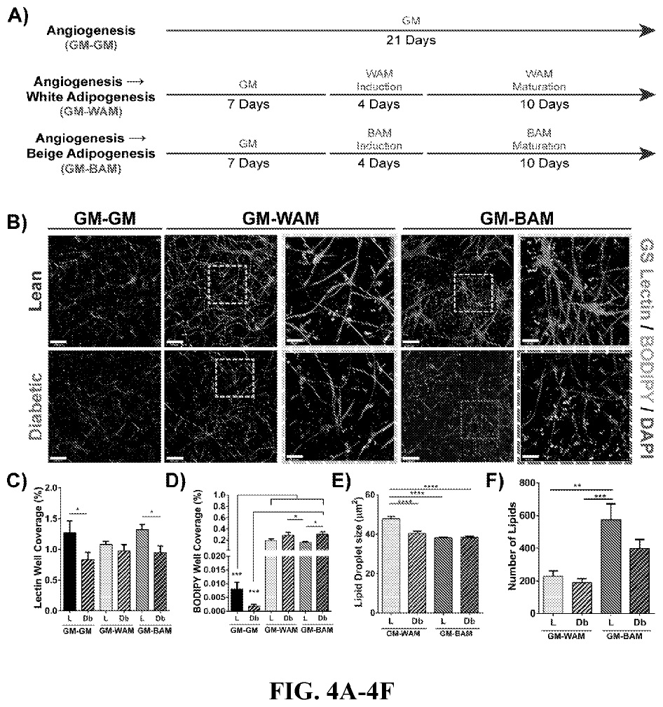

Formation of vascularized adipose tissue. Vascularization is critical to the development of adipose tissue. It is well-established that MVFs can be stimulated to assemble into microvascular networks. However, it is unclear if the precursor cells retain their differentiation capacity following network formation. MVFs from lean and diabetic animals were stimulated for 7 days prior to exposure to either white or beige adipogenic media for 14 days (GM-WAM or GM-BAM). Control scaffolds consisted of MVFs formed after culture in growth media for 21 days (GM-GM). The tissue constructs were first qualitatively examined through the confocal images of the immunofluorescence staining at 21 days (FIG. 4). Both lean and diabetic MVF grown for 21 days in control conditions demonstrated extensive vessel network formation. However, the vessel structures appeared smaller and less connected in the MVFs isolated from diabetic animals. The lean MVFs cultured for 21 days in growth media for 7 days prior to differentiation (GM-WAM and GM-BAM) exhibited branched vascular network formation together with clustered lipid droplets throughout the scaffold. On the other hand, the diabetic MVFs cultured in the same conditions exhibited reduced vessel formation, particularly in the GM-BAM group. Lipid droplet accumulation appeared similar to lean. In general, the clusters of lipid droplets were present in close proximity to the vessels.

Quantitative analysis of the images was performed by evaluating the percent of vascular and lipid staining within the scaffolds (FIG. 4C-D). The percent lectin staining, a measure of network formation, was at similar levels for the lean MVFs under all three culture conditions (1.3±0.2%, 1.1±0.05%, and 1.3±0.08%, respectively). Similarly, the percent of lectin staining in diabetic groups was comparable levels in the three growth conditions (0.83±0.1%, 0.97±0.1%, and 0.95±0.1%, respectively). However, the vascular network formation in the scaffolds from lean MVFs were generally higher than in diabetic MVFs. The lean control and GM-BAM groups exhibit a significantly higher percent of angiogenesis in respect to the diabetic MVFs (p=0.03 and p=0.05, respectively, FIG. 4C).

There was a statistically significant higher percent of BODIPY staining in lean and diabetic MVFs grown in GM-WAM and GM-BAM relative to control groups. In general, the diabetic MVFs had greater BODIPY staining in both GM-WAM and GM-BAM conditions relative to the lean MVFs. There was a statistically significant difference between the diabetic GM-BAM group and the lean GM-BAM scaffolds (0.1±0.01% vs. 0.3±0.04%, p=0.01, FIG. 4D). The size of lipid droplets in the GM-BAM conditions were significantly smaller than lean MVF in GM-WAM (38.2±0.4 μm², 38.4±0.5 μm² vs. 47.9±0.4 μm², p<0.0001, FIG. 4E). There was significantly greater number of lipid droplets in the lean MVF in GM-BAM compared to the lean and diabetic MVF in GM-WAM (575±96 vs. 229±33, p=0.002 and vs. 190±23, p=0.0005, FIG. 4F).

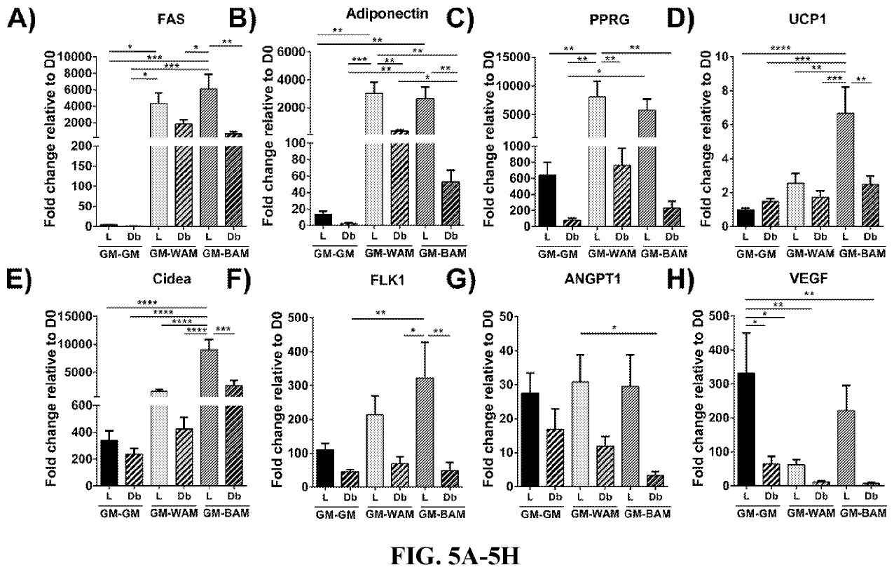

Gene expression by the vascularized tissues was analyzed as described above. FAS, adiponectin, and PPRG all showed similar trends of higher expression levels in lean MVFs compared to diabetic MVFs (FIG. 5A-C). Both thermogenic markers, UCP1 and Cidea, showed their highest expression in the lean MVFs exposed to GM-BAM conditions. UCP1 expression levels in lean MVFs grown in GM-BAM conditions was statistically increased relative to all other experimental groups (FIG. 5D). Similarly, the lean MVF in GM-BAM showed a statistically significant increase in Cidea expression levels compared to all other groups. Cidea expression levels were increased by ˜6 fold in lean GM-BAM relative to GM-WAM (FIG. 5E). These findings confirm that the MVFs isolated from both lean and diabetic rats retain their ability to express beige adipose markers following vascularization. Overall, MVF isolated from lean rats exposed to both GM-WAM and GM-BAM conditions exhibited higher expression of genes associated with adipogenesis and thermogenesis than MFV isolated from diabetic rats.

Similar to results with direct adipogenic differentiation, the angiogenic genes FLK1, ANGPT1, and VEGF were expressed at higher levels in vascularized tissues derived from lean MVFs relative to diabetic MVFs under all three differentiation conditions (FIG. 5F-H). FLK1 expression in the lean GM-BAM group (˜322 fold) was significantly higher than MVF from diabetic animals (˜48 fold, p=0.007, FIG. 5F). ANGPT1 showed similar expression levels in the lean MVFs across the three growth conditions (˜27 fold, ˜30 fold, and ˜29 fold, respectively) and were higher relative to the diabetic MVFs (FIG. 5G). The highest VEGF expression was in the lean GM-GM group (˜331 fold), which was significantly higher relative to all experimental groups except lean MVF in GM-BAM conditions (FIG. 5H). Like the other angiogenic markers, the diabetic GM-BAM group had the lowest expression levels of VEGF, which agrees with the reduced vessel network formation observed in the immunofluorescence staining.

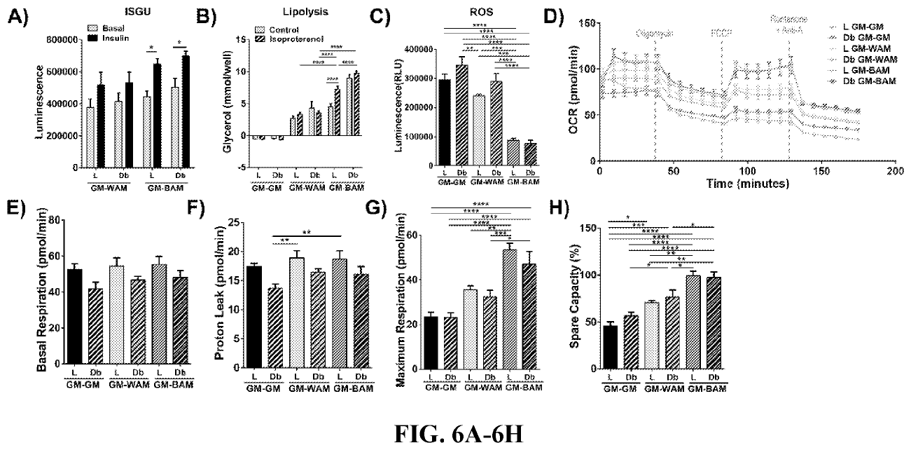

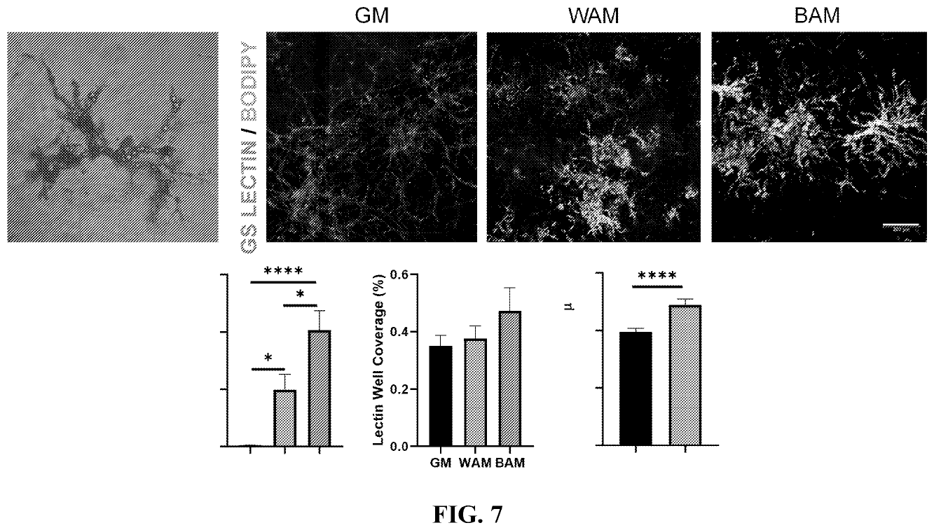

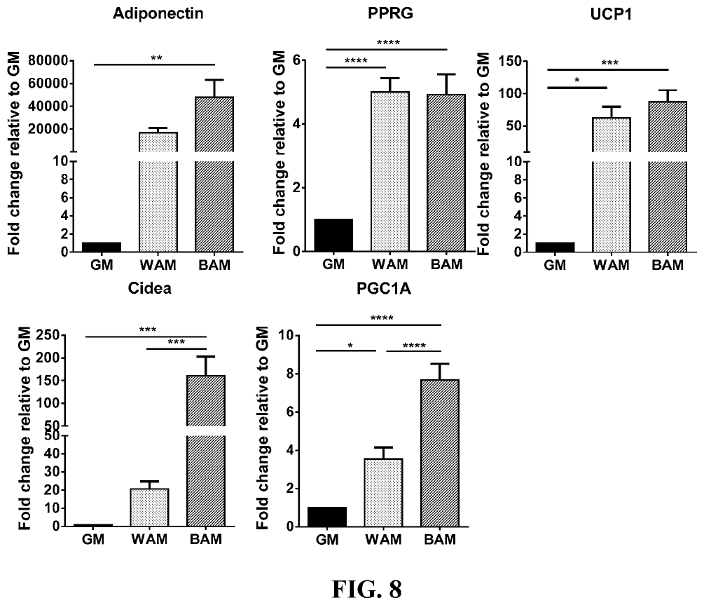

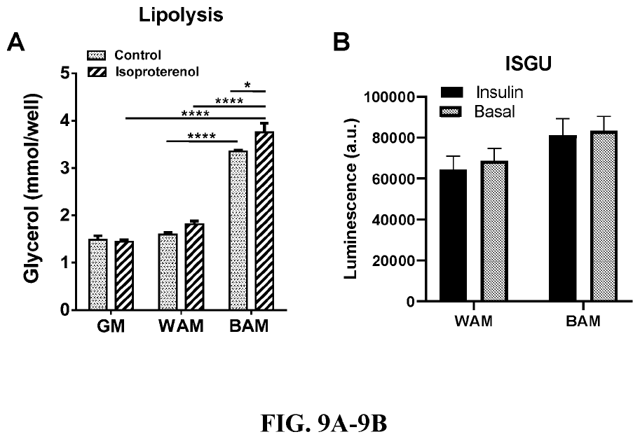

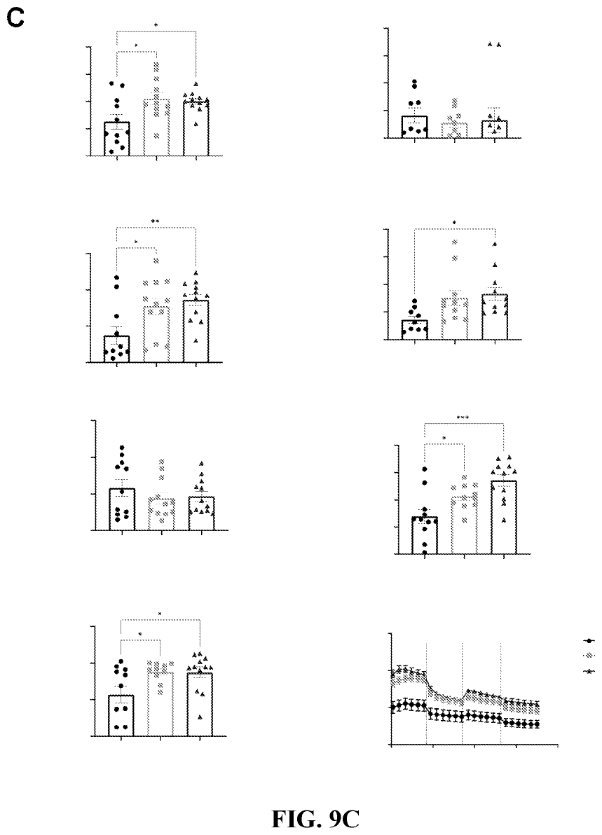

Functional assessment of the engineered vascularized adipose scaffolds. Functional testing was done on the vascularized adipose scaffolds. All experimental groups exhibited increased glucose uptake after insulin stimulation (FIG. 6A). In addition, glucose uptake in the vascularized tissues exposed to GM-BAM condition was higher than those exposed to GM-WAM with MVF from both lean and diabetic animals. There was an overall trend of increased lipolysis in GM-BAM groups in MVFs from both lean and diabetic animals relative to GM-WAM (FIG. 6B). There was a significant increase in glycerol in the lean MVF in GM-BAM conditions following isoproterenol stimulation (7.3±0.5 mmol/well vs. 4.5±0.5 mmol/well, p<0.0001). Additionally, vascularized tissues from lean MVF for cultured in GM-BAM contained significantly higher glycerol levels relative to GM-WAM from lean and diabetic animals (7.3±0.5 mmol/well vs. 3.4±0.3 mmol/well and 3.6±0.3mmol/well, respectively, p<0.0001) when stimulated with isoproterenol. Notably, isoproterenol stimulation resulted in higher glycerol levels in all adipogenic groups except diabetic MVFs in GM-WAM. Increased UCP1 expression in brown or beige adipocytes is expected to result in reduced production of reactive oxygen species (ROS). ROS levels in vascularized tissues derived from MVF from both lean and diabetic animals and exposed to GM-BAM were significantly lower than GM-GM and GM-WAM (FIG. 6C).

The metabolic activity of the vascularized adipose tissue models, was evaluated by quantifying oxygen consumption rate (OCR) with exposure to oligomycin, FCCP, and rotenone/antimycin A in sequence over the course of 3 hours (FIG. 6D). Basal respiration and proton leak showed similar trends, with lean MVFs exhibiting slightly higher values than diabetic MVFs under all three conditions, but there were no significant differences in basal respiration (FIG. 6E-F). Maximum respiration, on the other hand, was higher in lean and diabetic MVFs in GM-BAM exposure relative to GM-GM and GM-WAM (FIG. 6G). The maximum respiration was highest in lean MVFs in GM-BAM (53.5±2.9 pmol/well) which was significantly greater than MVFs in GM-GM (23.4±2.1 pmol/well and 23.2±2.1 pmol/well for lean and diabetic, p<0.0001) and GM-WAM (35.5±1.9 pmol/well, p=0.002 for lean and vs. 32.4±2.8 pmol/well, p=0.0002 for diabetic). Finally, percent spare capacity was calculated from the OCR data as a measure of the maximum respiration capability that cells have upon energetic demand (FIG. 6H). Both lean and diabetic MVFs in GM-BAM had a significantly greater percent spare capacity relative to MVF in GM-GM and GM-WAM. These data indicate that lean and diabetic MVF in GM-BAM conditions were more metabolically active than the scaffolds exposed to either GM-WAM or GM-GM.

### B. Materials and Methods

This study was conducted in compliance with the Animal Welfare Act and the Implementing Animal Welfare Regulations in accordance with the principles of the Guide for the Care and Use of Laboratory Animals. All animal procedures were approved by the Institutional Animal Care and Use Committee at the University of Texas at San Antonio.

Animals. Obese (FA/FA) and lean (FA/+) male Zucker diabetic fatty (ZDF) rats were obtained from Charles River (Wilmington, MA). The ZDF rats were acquired at 4 weeks of age and fed Purina 5008. Diabetes was confirmed in the FA/FA rats based on glucose tests of blood drawn from the lateral saphenous vein. Glucose levels greater than 300 mg/dL was selected as an indicative parameter of the onset of diabetes. All animals were housed in a temperature-controlled environment with a 12-h light-dark cycle and fed ad libitum.

Tissue Harvest and Microvascular Fragment Isolation. Lean and diabetic rats at 15-19 weeks of age were sacrificed and both anterior and posterior subcutaneous (SubQ) adipose tissue depots harvested. Microvascular fragments (MVFs) were isolated from the subcutaneous fat depots similar to that previously described (Stone and Rathbone, Global open 4(12), 2016, e1140). Briefly, the adipose tissue was incubated in collagenase type I (6 mg/ml, Worthington Biochemical Corporation, Lakewood, NJ) at 37° C. with agitation for 8-15 minutes, based on visualization of digestion level. The digested material was centrifuged (400 g×4 min) resulting in a floating layer of adipocytes and a pellet containing a heterogeneous mixture of cells and MVFs. The pellet was resuspended in phosphate-buffered saline (PBS) containing 0.1% bovine serum albumin (Sigma-Aldrich; St. Louis, Mo.) and filtered through 500 μm and 37 μm filters (Carolina Biological Supply, Burlington, NC) to remove large debris and minimize cell contamination, respectively. The collected MVFs were then counted and centrifuged for loading into the hydrogel precursor solution.

Scaffold formation and Culture Conditions. Fibrin scaffolds were formed by combining 20 mg/ml fibrinogen (Sigma-Aldrich; St. Louis, Mo.) containing 20,000 MVFs/mL in DMEM with 10 U/mL thrombin (MilliporeSigma, St. Louis, MO.) in 96-well culture plates. The hydrogels for gene expression analysis were 100 μL in volume while gels for all other analyses were 50 μL, except for OCR where 30 μL gels were used. MVFs used in these experiments were isolated from four different rats, two lean and two diabetic. All experimental conditions were repeated at least twice.

MVFs were cultured in growth media (GM; Dulbecco's Modified Eagle Medium (DMEM) containing 20% Fetal Bovine Serum, 1% Pen-Strep, and 0.2% MycoZap) for 7, 14, or 21 days. To stimulate adipogenesis, MVFs were grown in white adipogenic media (WAM) which consisted of a 4 day treatment with induction media (DMEM/F12 containing 20% Fetal Bovine Serum, 1% Pen-Strep, 0.2% MycoZap, 10 μg/ml Insulin, 10 μM Forskolin and 1 μM Dexamethasone) followed by maintenance media (DMEM/F12, 20% Fetal Bovine Serum, 1% Pen-Strep, 0.2% MycoZap and 5 μg/ml Insulin) for 10 days. Alternatively, MVFs were grown in beige adipogenic media (BAM) conditions which consisted of a 4 day treatment with induction media (WAM with added 1 μM Rosiglitazone, and 20 nM T3) followed by maintenance media (WAM with added 10 μM Forskolin, 1 μM Rosiglitazone, and 20 nM T3) for 10 days. Further, to engineer vascularized beige adipose constructs, the MVF fibrin scaffolds were initially grown in GM for 7 days (pre-sprouting) followed by 14 days of either WAM or BAM conditions. For all treatments the medium (100 μL in a 96-well plate) was replaced every other day throughout the study while cultures were maintained in a humidified incubator at 37° C. and 5% CO₂.

RNA isolation and quantitative RT-PCR. RNA was isolated from fibrin scaffolds (100 μL) containing MVFs (n=4 individual hydrogels/group) and purified using a Qiagen RNeasy Mini Kit (Valencia, CA) according to manufacturer guidelines. mR.NA concentrations were measured using a Take3 Micro-Volume Plate (BioTek, Winooski, VT), then normalized to 150 ng of mRNA for conversion to cDNA. The isolated RNA was converted to cDNA using the iScript cDNA synthesis kit (BioRad, Hercules, CA). Real-time quantitative polymerase chain reaction (qPCR) was performed using a CFX96 Touch Real-Time PCR Detection System (BioRad, Hercules, CA). All primers used to carry out the analysis were predesigned primers (Sigma-Aldrich; St. Louis, Mo). Ten μL of iTaq Universal SYBR Green Supermix (BioRad, Hercules, CA) was used for each reaction. Fold expression levels were calculated using the 2^{-ΔΔCt} method, where the GM gels at day 1 were designated as the calibrator group and GAPDH expression was used as the endogenous control (Livak and Schmittgen, Methods 25(4), 2001 402-8).

Lipolysis Assay. A lipolysis assay measuring glycerol release was completed using the Lipolysis Colorimetric Assay Kit according to manufacturer's recommendations (BioVision, Milpitas, CA) with a few modifications. Briefly, at the end of the differentiation protocol, gels (n=12/group) were washed two times with provided Lipolysis Wash Buffer, which was then replaced with the Lipolysis Assay Buffer. Ten uM Isoproterenol (final concentration 100 nM) was added to half the wells to stimulate lipolysis for 3 hr. Following stimulation, 50 μL of media was collected into a 96-well plate and 50 μL reaction mix, provided by the manufacturer, was added and incubated at room temperature for 1 hour, after which absorbance was read at OD 570 nm, with the amount of glycerol released calculated using a standard curve.

Insulin Stimulated Glucose Uptake Assay. Insulin-stimulated glucose analysis was performed according to the manufacturer's instructions (Glucose Uptake-Glo™ Assay, Promega, Madison, WI), with some modifications. Briefly, MVF scaffolds were cultured in DMEM without serum or glucose for 24 hours. Afterwards, samples were changed to DMEM±insulin (1 mM) for 2 hours, followed by the addition of 2-Deoxyglucose (0.1 mM) for 1 hour. Finally, a 2-Deoxyglucose-6-phosphate (2DG6P) detection reagent was used to quantify the amount of glucose internalized by the cells. Luminescence was measured after 2 hours with a spectrophotometer (Biotek, Vinooski, VT) (n=6/group).

Immunofluorescence analysis. Hydrogels were fixed in 4% formaldehyde for 2 hours at room temperature, permeabilized using 0.5% Triton-X for 20 min, blocked using 10% goat serum for 2 hours, then stained using Rhodamine labeled Griffonia (Bandeiraea) Simplicifolia Lectin I (GS-1; Vector Labs, Burlingame, CA, 1:100), boron-dipyrromethene (BODIPY; ThermoFisher, D3922, 1:100), and DAPI (ThermoFisher, R37606). The distribution of both vessels and lipid droplets of entire wells (n=6/group) were determined using a Leica TCS SP8 Confocal Microscope (Buffalo Grove, IL) using a rendering of 100 μm thickness/10 μm per section of the entire well. Quantification was performed using the Leica 3D analysis toolkit with Otsu thresholding.

ROS Assay-Hydrogen Peroxide Assay. The ROS-Glo H2O2 Assay uses a modified luciferin substrate, based on boronate oxidation, which reacts directly with hydrogen peroxide (H₂O₂) to generate a luciferin precursor. Upon addition of detection reagent, the precursor is converted to luciferin and Ultra-Glo Recombinant Luciferase included in the detection reagent produces a light signal proportional to the level of H₂O₂ in the sample. A mixture of the H₂O₂ substrate and H₂O₂ dilution buffer (150 μl) was added to each well containing the scaffolds. Six hours later, 50 μl of the media was mixed with 50 μl of ROS-GLO Detection Solution in a separate plate and incubated at room temperature for 20 min. Luminescence was read using a Take3 Micro-Volume Plate (BioTek, Winooski, VT).

OCR analysis. A mitochondrial stress test was performed to analyze the cellular metabolic activity of the differentiated 3D culture models (n=10) using a Seahorse XFe96 Flux Analyzer (Seahorse Bioscience). The XF Analyzer measures the oxygen consumption rate (OCR) of live cells in real-time. The sequential application of drugs that inhibit different electron transport chain enzymes (ETC) is utilized to determine metabolic parameters such as basal and maximal cellular respiration, proton leak, and spare capacity (Ferrick et al., Drug Discov Today 13(5-6), 2008, 268-74).

At the start of the assay, an initial OCR reading was performed. Oligomycin was added to the culture wells to inhibit ATP synthase, reducing the contribution of ATP production and revealing proton leak in the ETC (after correcting for nonmitochondrial oxygen consumption). Second, the ionophore carbonyl cyanide-ptrifluoromethoxyphenylhydrazone (FCCP) was applied to induce maximum respiration. Finally, rotenone and antimycin A were used to inhibit the ETC enzymes upstream of oxygen consumption, thus eliminating all mitochondrial oxygen expenditure. The final OCR represents the background nonmitochondrial oxygen consumption of the cells. Subtracting the final OCR after rotenone/antimycin A treatment from initial OCR before oligomycin treatment gives the basal mitochondrial metabolism or the mitochondrial OCR at resting state (Dranka et al., Free Radic Biol Med 51(9), 2011, 1621-35).

For OCR measurement, fibrin gels (30 μL), as described previously, were seeded into XF96 V3 PS Tissue Culture Microplates and cultured for either 14 or 21 days (based on groups as described previously), cells were then placed in a 37° C. incubator without CO₂ for 45 min before the assay. After basal measurements, oligomycin (1 mM), FCCP (250 nM), and rotenone/antimycin A (2 mM/2 mM) were injected sequentially to characterize the mitochondrial function of the cells. Six measurements were taken before and following the application of each drug solution. Oligomycin, FCCP, and rotenone/antimycin A were injected into the medium at 42, 84, and 126 min, respectively.

Statistical Analysis. Graphpad Prism Software 7 (GraphPad Software, Inc., La Jolla, CA) was used to run one, or two-way analysis of variance (ANOVA) tests with Holm-Sidak's multiple comparison analyses to determine differences between groups. Statistical significance was defined as p<0.05. All results are presented as mean±standard error of the mean (SEM).

1. A thermogenic composition comprising thermogenic cells in a vascularized hydrogel, the thermogenic cells being produced by culturing microvascular fragments in a thermogenic differentiation media.

2. The composition of claim 1, wherein the thermogenic differentiation media comprises one or more of insulin, forskolin, dexamethasone, rosiglitazone, and T3.

3. The composition of claim 1, wherein the thermogenic cells are characterized by one or more of (a) multilocular lipid droplets, (2) increased number of mitochondria, (3) increased oxygen consumption rate, (4) increase in mitochondrial respiration, and (5) increased metabolic activity compared to undifferentiated microvascular fragments.

4. The composition of claim 1, wherein the thermogenic cell expresses uncoupling protein 1 (UCP1) mRNA at a level that is at least 150% as compared to UCP1 mRNA expression in undifferentiated MVFs.

5. The composition of claim 1, wherein the thermogenic cell expresses cell death-inducing DNA fragmentation factor alpha-like effector A (Cidea) mRNA at a level that is at least 150% as compared to Cidea mRNA expression in undifferentiated MVFs.

6. The composition of claim 4 or claim 5, wherein mRNA level is determined by reverse transcriptase quantitative polymerase chain reaction (RT-qPCR).

7. The composition of claim 1, wherein the UCP1 and Cidea protein levels are 200% as compared to protein levels in undifferentiated MVFs.

8. The composition of claim 7, wherein the protein level is determined by Western blot.

9. The composition of claim 1, wherein maximum respiration of the thermogenic cells as measured by oxygen consumption rate (pmol/min) after exposure to the ionophore carbonyl cyanide-ptrifluoromethoxyphenylhydrazone (FCCP) is at least 150% as compared to undifferentiated MVFs.

10. The composition of claim 1, wherein isoproterenol stimulated lipolysis as measured by glycerol release of the thermogenic cells is at least 12.5 mmol glycerol/cm².

11. The composition of claim 1, wherein spare capacity of the thermogenic cells is at least 160% as compared to undifferentiated MVFs.

12. The composition of claim 1, wherein the hydrogel comprises 10 U/mL thrombin to 20 mg/mL fibrinogen.

13. The composition of claim 1, wherein differentiation of thermogenic cells comprises growing MVFs in a growth media for 0-14 days followed by growing the MVFs in the thermogenic differentiation media.

14. A method of producing a thermogenic composition comprising the steps of:
mixing microvascular fragments (MVFs) isolated from a tissue in a hydrogel precursor solution;
forming a MVF hydrogel by mixing the hydrogel precursor solution with fibrinogen and thrombin to form a MVF hydrogel;
culturing the MVF hydrogel in a differentiation media containing one or more of insulin, forskolin, dexamethasone, rosiglitazone, or T3 forming a cultured thermogenic hydrogel

15. The method of claim 14, wherein the tissue is adipose tissue.

16. The method of claim 14, further comprising:
isolating MVF by incubating a tissue sample with collagenase forming a tissue digest;
centrifuging the tissue digest forming a pellet and a floating layer;
resuspending the pellet forming a suspension and filtering the suspension to collect MVFs in the filtrate forming collected MVFs.

17. The method of claim 14, wherein the thermogenic hydrogel is cultured for 5 to 15 days.

18. The method of claim 14, wherein the growth media comprises Dulbecco's Modified Eagle Medium (DMEM) containing 20% Fetal Bovine Serum (FBS), 1% Penicillan-Streptomycin, and 0.2% MycoZap.

19. The method of claim 14, wherein the differentiation media contains one or more of 10 μg/ml Insulin, 10 μM Forskolin, 1 μM Dexamethasone, 1 μM Rosiglitazone, or 20 nM T3.

20. The method of claim 14, wherein the differentiation media contains 10 μg/ml Insulin, 10 μM Forskolin, 1 μM Dexamethasone, 1 μM Rosiglitazone, and 20 nM T3.

21. The method of claim 20, wherein the cultured MVF hydrogel is cultured for 2 to 21 days.

22. The method of claim 21, wherein the thermogenic MVF is cultured in a maintenance media.

23. The method of claim 22, wherein the maintenance media comprises DMEM/F12 containing 20% Fetal Bovine Serum (FBS), 1% Penicillan-Streptomycin, 0.2% MycoZap, 10 μg/ml Insulin, 10 μM Forskolin, 1 μM Rosiglitazone, and 20 nM T3.

24. An isolated thermogenic microvascular fragment (MVF) produced by the methods of claim 14.

25. The thermogenic MVF of claim 24, wherein expression of thermogenic gene UCP-1 is increased and/or lipolysis increases upon exposure to isoproterenol as compared to an undifferentiated MVF.

26. A method of treating obesity or a metabolic disease comprising implanting the engineered thermogenic MVF of claim 24 into a subject in need thereof.
